(19) United States
(12) Patent Application Publication
(10) Pub. No.: US 2025/0025859 A1
(43) Pub. Date: Jan. 23, 2025

(54) CATALYST COMPOSITIONS AND METHODS FOR DECOMPOSING FORMALDEHYDE THEREOF

(71) Applicant: RHT LIMITED, Hong Kong (CN)

(72) Inventors: Chi Keung Rudy CHAN, Hong Kong (CN); Ka Wai JIM, Hong Kong (CN); Lam Lung YEUNG, Hong Kong (CN)

(21) Appl. No.: 18/713,191

(22) PCT Filed: Nov. 21, 2022
(86) PCT No.: PCT/CN2022/133084

§ 371 Date: May 24, 2024

Related U.S. Application Data
(60) Provisional application No. 63/283,207, filed on Nov. 24, 2021.

(51) Int. Cl. B01J 23/34; B01D 53/00; B01D 53/86; B01D 53/88; B01J 21/04; B01J 29/06; B01J 35/39; B01J 35/40; B01J 35/64

(52) U.S. Cl. CPC B01J 23/34; B01D 53/007; B01D 53/8668; B01D 53/885; B01J 21/04; B01J 29/06; B01J 35/39; B01J 35/40; B01J 35/643; B01J 35/647; B01D 2255/2073; B01D 2255/802; B01D 2257/708; B01D 2258/06; B01D 2259/804

(57) ABSTRACT

In certain embodiments, one aspect provides a catalyst composition comprising a porous material (granules) having a plurality of nanopores and comprising one or more of silicon dioxide, aluminum oxide, and zeolite; and manganese oxides comprising manganese in an amount of about 0.1-50% by weight of the total catalyst composition.

### FIELD OF INVENTION

This invention relates to the field of catalyst compositions, processes for preparing the compositions and methods of using the compositions.

### BACKGROUND OF INVENTION

Formaldehyde is a common air pollutant which has been identified to be carcinogenic and teratogenic to human health by International Agency for Research on Cancer (IARC). It was reported that formaldehyde could be emancipated from many sources such as furniture, building materials, cooking fume and tobacco smoke. The recommendation of the World Health Organization of 30 min average concentration for formaldehyde is 0.1 mg/m³. Other volatile organic compounds (VOCs) in the air are also harmful to human health. Therefore, effective abatement of formaldehyde and other VOCs is urgently needed to meet human health needs and international environmental requirements.

### SUMMARY OF INVENTION

Provided herein are novel catalyst compositions and processes for preparing the catalyst compositions useful in removing volatile organic compounds (VOCs), such as formaldehyde, from the air.

One aspect provides a catalyst composition comprising a porous material having a plurality of nanopores and manganese oxides comprising manganese in an amount of about 0.1-50% by weight of the total catalyst composition.

Another exemplary embodiment provides a catalyst composition comprising a porous material having a plurality of nanopores and comprising one or more of silicon dioxide, aluminum oxide, and zeolite; and manganese oxides comprising manganese in an amount of about 0.1-50% by weight of the total catalyst composition.

In a further aspect, the manganese oxides form one or more clusters on the porous material. In some embodiments, the manganese oxides are selected from the group consisting of MnO, MnO₂, MnO₃, Mn₃O₄ and Mn₂O₃. In some embodiments, the manganese is equal to or less than 20% by weight of the total catalyst composition.

In a further aspect, the zeolite comprises one or more aluminum oxides and silicon oxides. In some embodiments, the aluminum oxide comprises one or more amorphous aluminum oxide, crystalline aluminum oxide, activated aluminum oxide.

In a further aspect, the porous material is provided in the form of a granule. In other embodiments, the diameter of each granule is 3-5 mm. In some embodiments, the manganese oxides are added to the granules via doping, ion exchange, or deposition. In some embodiments, the manganese oxides have total bandgap energy of 2.0 eV to 3.75 eV.

In certain embodiments, provided is a method of decomposing formaldehyde comprising the steps of: (a) activating the catalyst composition with Vacuum Ultraviolet (VUV) to generate an activated catalyst composition comprising reactive oxygen species (ROS); and (b) subjecting the activated catalyst composition comprising the ROS to formaldehyde to form carbon dioxide and water. In some embodiments, the VUV is generated by a VUV lamp having an output of 4.7 W with a wavelength of 185 nm or 29.7 W with a wavelength of 254 nm.

In certain embodiments, provided is a process for preparing the catalyst composition, comprising: (a) adding a porous material to a manganese salt solution to form a mixture; and (b) calcinating the porous material, wherein at least one nano sheet of manganese oxides is formed on the porous material.

In certain embodiments, the porous material is zeolite, aluminum oxide, or silica gel. In some embodiments, the zeolite is aluminum zeolite, analcime, chabazite, clinoptilolite, heulandite, phillipsite, stilbite, or natrolite.

In certain embodiments, the process further comprises washing the porous material with water and/or acid before adding the porous material to the manganese salt solution. In some embodiments, the process further comprises drying the porous material on a glass tray after washing the porous material before adding the porous material to the manganese salt solution. In some embodiments, the process further comprises weighing the dried porous material and the manganese salt after drying the porous material. In some embodiments, the process further comprises stirring the mixture after adding the porous material to the manganese salt solution. In some embodiments, the process further comprises filtering the porous material after adding the porous material to the manganese salt solution. In some embodiments, the process further comprises cooling down the porous material to room temperature after calcinating the porous material.

Some embodiments provide an air purification system comprising an air blower; at least one chamber having an outlet for air to flow out of the chamber; and a catalyst composition described herein. In some embodiments, the air purification system is a grossing station. In some embodiments, the air purification system has a removal efficiency for formaldehyde in the chamber of at least 95% within 20 minutes. In some embodiments, the air purification system removes formaldehyde in the chamber at a rate such that the total concentration of formaldehyde in the chamber is 1-2 ppm within 20 minutes. In some embodiments, the air purification system further comprises at least one VUV lamp. In some embodiments, the air purification system further comprises an ozone/ion generator.

### Advantages of the Current Invention

In certain embodiments, the disclosed compositions and methods have several advantages over the current solutions. For example, current methods for preparing the catalyst composition of manganese-doped porous materials require high temperature, such as more than 500° C. These techniques require harsh conditions for the industry and thus are not efficient and can be dangerous when bulk production of the catalyst composition is needed. Certain embodiments of the currently claimed method do not require such a high temperature but can still efficiently produces the catalyst composition.

Additionally, in certain embodiments, the disclosed methods provide the catalyst composition of manganese-doped porous materials in surprisingly high yield and high quality, despite adding a step of filtering the mixture of the porous material and manganese salt solution before calcinating the same. This is surprisingly better over other methods for obtaining the catalyst composition, such as drying or boiling the mixture without filtering. The currently claimed method enhances the quality of the catalyst composition compared to the previous methods.

Other example embodiments are discussed herein.

### BRIEF DESCRIPTION OF FIGURES

FIG. 1A is a ball-and-stick diagram which illustrates a top view of an example embodiment of manganese oxides doped on the silica materials.

FIG. 1B is a ball-and-stick diagram which illustrates a side view of said example embodiment of manganese oxides doped on the silica materials.

FIG. 2 is a schematic diagram which illustrates an example embodiment of how MnOx serves as a photocatalyst for removing organic pollutants.

FIG. 3 is a flowchart which illustrates a method for preparing a catalyst composition in accordance with an example embodiment.

FIG. 4 is a diagram which illustrates an example embodiment of a volatile organic compound (VOC) removal testing unit and its components for removing VOC.

FIG. 5 is a photo showing an example embodiment of a grossing station.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) are used, this disclosure/application also includes alternate embodiments where the term “comprising,” “including,” or “containing,” is replaced with “consisting essentially of” or “consisting of.” These alternate embodiments that use “consisting of” or “consisting essentially of” are understood to be narrower embodiments of the “comprising,” “including,” or “containing” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising,” including, and “containing,” and any related forms are open-ended terms which allow for additional elements or features beyond the named essential elements, whereas “consisting of” is a closed-end term that is limited to the elements recited in the claim and excludes any element, step, or ingredient not specified in the claim.

As used herein, the singular forms “a,” “an” and “the” are intended to include the plural forms as well, unless the context clearly indicates otherwise. Where a range is referred to in the specification, the range is understood to include each discrete point within the range. For example, 1-7 means 1, 2, 3, 4, 5, 6, and 7.

As used herein, “Nano Confined Catalytic Oxidation (NCCO)” refers to the technology in which active oxygens are emitted from a generator and kill bacteria and viruses, as well as destroy the molecular structure of harmful chemicals.

As used herein, “zeolite” refers to microporous, aluminosilicate minerals commonly used as commercial adsorbents and catalysts.

As used herein, “vacuum ultraviolet (VUV)” refers to ultraviolet light with a wavelength shorter than 200 nm.

As used herein, “nano sheet layer” refers to a few layers of the given materials. In certain embodiments, it refers to a single layer of the manganese oxides formed on the porous material, but it can also refer to a few layers of the manganese oxides.

As used herein, “deposition” refers to depositing a particular material onto the surface of another material. In certain embodiments, it refers to depositing manganese oxides on porous materials.

As used herein, “doping” refers to the introduction of impurities into material for the purpose of modulating its properties. In some embodiments, doping refers to adding small amounts of manganese oxides into porous materials.

As used herein, “ion exchange” refers to a reversible interchange of one kind of ion present on an insoluble solid with another of similarly charged ion present in a solution surrounding the solid.

As used herein, “overnight” refers to a few hours that cover the duration of a night. In some embodiments, this is 5-24 hours, 5-18 hours, or 5-12 hours.

As used herein, “calcination” refers to a process of heating of solids to high temperature for the purpose of removing volatile substances, oxidizing a portion of the mass, or rendering them friable.

As used herein, “volatile organic compounds (VOCs)” refers to volatile organic compounds that have a high vapor pressure and low water solubility and are found in a wide variety of products such as solvent-based paints, printing inks, many consumer products, organic solvents and petroleum products. Examples include but are not limited to formaldehyde, methylene chloride, benzene, acetone, perchloroethylene, ethylene glycol, tetrachloroethylene, toluene, xylene and 1,3-butadiene.

Although the description referred to particular embodiments, the disclosure should not be construed as limited to the embodiments set forth herein.

### Catalyst Composition

In certain exemplary embodiments, the catalyst composition comprises a porous material having a plurality of nanopores and comprising one or more of silicon dioxide, aluminum oxide, and zeolite; and manganese oxides comprising manganese in an amount of about 0.1-50% by weight of the total catalyst composition.

In one exemplary embodiment, the zeolite comprises one or more aluminum oxides and silicon oxides. In some embodiments, the porous material is aluminum oxide. In some embodiments, the aluminum oxide comprises one or more amorphous aluminum oxide, crystalline aluminum oxide, activated aluminum oxide.

In certain exemplary embodiments, manganese oxides form one or more clusters on the porous material. In some embodiment, the manganese has an oxidation state of +2, +3, +4, +5 or +6.

In one exemplary embodiment, the manganese oxides are selected from the group consisting of MnO, MnO₂, MnO₃, Mn₃O₄ and Mn₂O₃. In some embodiments, the manganese is equal to or less than 20% by weight of the total catalyst composition.

In certain embodiments, the manganese is 1-20% by weight of the total catalyst composition. In other exemplary embodiments, the manganese is 2-20% by weight, 3-20% by weight, 4-20% by weight, 5-20% by weight, 6-20% by weight, 7-20% by weight, 8-20% by weight, 9-20% by weight, 10-20% by weight of the total catalyst composition.

In other exemplary embodiments, the manganese is 10.78% by weight of the total catalyst composition.

In certain embodiments, the zeolite comprises one or more aluminum oxides and silicon oxides. In some embodiments, the porous material is aluminum oxide. In some embodiments, the aluminum oxide comprises one or more amorphous aluminum oxide, crystalline aluminum oxide, and activated aluminum oxide.

In certain embodiments, the zeolite includes fibrous zeolites, wherein the fibrous zeolites include gonnardite, natrolite, mesolite, paranatrolite, scolecite, tetranatrolite, edingtonite, kalborsite, and thomsonite-series. In some embodiments, the zeolite includes the zeolites with chains of single connected 4-membered rings, including but not limited to analcime, leucite, pollucite, wairakite, laumontite, yugawaralite, goosecreekite, and montesommaite. In some embodiments, the zeolite includes the zeolites with chains of doubly connected 4-membered rings, including but not limited to harmotome, phillipsite-series, amicite, gismondine, garronite, gobbinsite, boggsite, merlinoite, mazzite-series, paulingite-series, and perlialite. In some embodiments, the zeolite includes the zeolites with chains of 6-membered rings, including but not limited to chabazite-series, herschelite, willhendersonite, SSZ-13, faujasite-series, Linde type X, Linde type Y, maricopaite, mordenite, offretite, wenkite, bellbergite, bikitaite, erionite-series, ferrierite, gmelinite, levyne-series, dachiardite-series, and epistilbite. In some embodiments, the zeolite includes the zeolites with chains of T₁₀O₂₀ tetrahedra (T=combined Si and Al), including but not limited to clinoptilolite, heulandite-series, barrerite, stellerite, stilbite-series, brewsterite-series. In some embodiments, the zeolite includes cowlesite, pentasil, tschernichite, and Linde type A framework.

In some embodiments, the catalyst compositions comprise manganese oxides and porous materials. The porous materials include granules silicon dioxides such as quartz, glass beads, silica gel or zeolites made up of aluminum oxides and silicon oxides with clear nano pore size (diameter) of 0.2-0.6 nm. In some embodiments, the nano pore size (diameter) of the nanopores is 0.2-0.4 nm. In some embodiments, the nano pore size is one or more of 0.1, 0.2, 0.3, 0.4, 0.5, or 0.6 nm.

In some embodiments, the catalyst composition is made by doping, ion exchange or deposition. Methods of doping, ion exchange, and deposition of manganese oxides on porous materials that are known in the art may be used.

In some embodiments, the silica gel is a clear pellet, having drying and moisture-proof properties. In some embodiments, the silica gel is a translucent white pellet and a liquid adsorbent. In some embodiments, the silica gel is translucent, has a micro-pored structure, or is a raw material used for the preparation of silica gel cat litter. In some embodiments, if additionally dried and screened, the silica gel forms macro-pored silica gel which is used as a drier, adsorbent and catalyst carrier. In some embodiments, the silica gel is in the form of a granule or a bead.

Silica gel has several advantages. In certain embodiments, silica gel provides good adhesion to manganese oxides. Silica gel is also inexpensive and mechanically robust.

In certain embodiments, the porous material is provided in the form of a granule. In some embodiments, the diameter of each granule is 3-5 mm. In some embodiments, the manganese oxides are added to the granule via doping, ion exchange or deposition.

In certain embodiments, the manganese oxides have a total band gap energy of 2.0 eV to 3.75 eV.

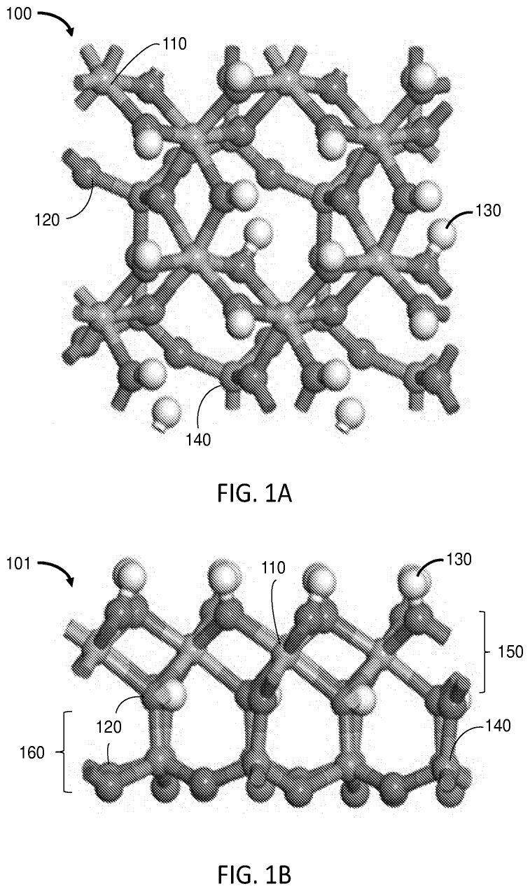

With reference to FIG. 1A and FIG. 1B, an example catalyst composition 100 may include a nano sheet 150 of manganese oxides formed on the silicon oxides. FIG. 1A is a ball-and-stick diagram that illustrates a top view of the catalyst composition 100. According to FIG. 1A, manganese 110 marked in purple and oxygen 120 marked in red are combined as manganese oxides and formed on a top layer of the catalyst composition 100. At the bottom layer, silicon 140 marked in yellow and oxygen 120 are combined as silicon oxides as the porous material according to an example embodiment. The silicon oxides serve as a platform on which the manganese oxides are formed. Hydrogen 130 molecules marked in white are bound to the oxygen 120 molecules.

FIG. 1B is a ball-and-stick diagram that illustrates a side view of the catalyst composition 100. According to FIG. 1B, a nano sheet layer 150 of the manganese oxides is formed on top of the bottom layer 160 of the silicon oxides. In some embodiments, the nano sheet layer 150 includes one or more layers of the manganese oxides on top of the porous materials.

### Activation of Catalyst Composition

In one exemplary embodiment, the catalyst composition is oxidized by reactive oxygen species (ROS) directly generated by vacuum ultraviolet (VUV). In some embodiments, ROS oxidizes manganese oxides with the help of the catalyst composition to its higher oxidation state which reacts with formaldehyde to form carbon dioxide and water. In some embodiments, the high valent manganese ion in the catalyst returns to its original oxidation state after the oxidation.

In certain embodiments, ROS generated by VUV can oxidize the manganese doped porous material according to the reactions below, which reacts with formaldehyde to form carbon dioxide and water:

A) Mechanism of ozone scavenger

O₃+*MnOx→O₂+O*MnOx

O*MnOx+O₃→O₂+O₂*MnOx

O₂*MnOx→O₂+*MnOx

B) Mechanism of Doped Manganese Oxide with ROS

ROS+*MnOx→O*MnOx

O*MnOx+H₂O+*MnOx→2(OH*MnOx)

OH*MnOx+ROS→HO₂*MnOx

O*MnOx+ROS→O₂*MnOx

In certain embodiments, species O*MnOx, O₂*MnOx, OH*MnOx and HO₂*MnOx can all react with formaldehyde to form carbon dioxide and water:

HCHO+*MnOx→HCHO*MnOx

HCHO*MnOx+2 O*MnOx→CO₂+H₂O+3*MnOx

HCHO*MnOx+O₂*MnOx→CO₂+H₂O+2*MnOx

HCHO*MnOx+2 OH*MnOx+H⁺→CO₂+2H₂O+2*MnOx

HCHO*MnOx+HO₂*MnOx→CO₂+H₂O+H++2*MnOx

In certain embodiments, VUV with a wavelength 185 nm and 254 nm can generate many different types of ROS, including O·, OH·, HO₂·, O₃ and H₂O₂, which can react with formaldehyde and re-generate the manganese doped silica material:

O₂+hv→2O·

H₂O+hv→H·+OH·

O·+H₂O→2OH·

O·+O₂→O₃

OH·+OH·→H₂O₂

H·+O₂→HO₂·

2HO₂·→H₂O₂+O₂

H₂O+hv→H·+OH·

In certain embodiments, the manganese oxides formed on the porous materials serve as the photocatalyst. In some embodiments, the bandgap energy of manganese dioxide nano sheets is about 2.34 eV. A mesoporous structure containing Mn₃O₄ and Mn₂O₃ was found to have bandgap energies of 2.46 eV and 3.18 eV after calcination and uncalcined, respectively. One exemplary embodiment provides nano sheets of manganese oxides with multiple oxidation states and possessing a large number of band gaps over a large range (from 2.0 eV to 3.75 eV), which can absorb not only VUV but also visible light for photocatalytic oxidation of formaldehyde.

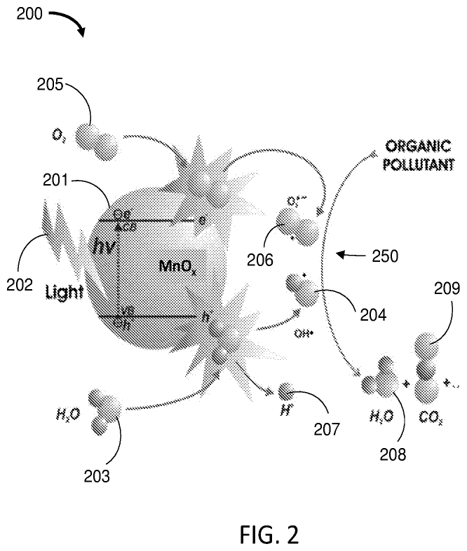

FIG. 2 is a schematic diagram 200 and example embodiment demonstrating how manganese oxides form on porous materials serving as a photocatalyst for removing an organic pollutant (e.g., formaldehyde). When MnOx 201 absorbs the energy from light 202, the oxidation state of the MnOx 201 becomes higher. In some embodiments, the light 202 can be VUV or visible light. H₂O 203 and O₂ 205 interact with MnOx 201 with the higher oxidation state to become OH. 204 and O₂· 206, respectively. H⁺ 207 is also generated when H₂O 203 reacts with MnOx 201 with a higher oxidation state. Finally, the organic pollutant becomes H₂O 208 and CO₂ 209 after the organic pollutant reacts with OH· 204 and O₂· 206.

One exemplary embodiment provides a method of decomposing formaldehyde comprising the steps of: (a) activating the catalyst composition with VUV to generate an activated catalyst composition comprising ROS; and (b) subjecting the activated catalyst composition comprising the ROS to formaldehyde to form carbon dioxide and water.

### Preparation of Catalyst Composition

One exemplary embodiment provides a process for preparing the catalyst composition comprising: adding a porous material to a manganese salt solution to form a mixture; and calcinating the porous material, wherein at least one nano sheet of manganese oxides is formed on the porous material.

In certain exemplary embodiments, the process for preparing the catalyst composition is useful for manufacturing the catalyst composition in bulk. In some embodiments, the porous material is zeolite, aluminum oxide, or silica gel. In some embodiments, the zeolite is aluminum zeolite, analcime, chabazite, clinoptilolite, heulandite, phillipsite, stilbite, or natrolite.

In certain exemplary embodiments, the aluminum zeolite is 50-150 g by weight. In some embodiments, the aluminum zeolite is approximately 110 g by weight.

In certain embodiments, the process may further comprise washing the porous material with water and/or acid before adding the porous material to the manganese salt solution. In certain embodiments, the process may further comprise drying the porous material on a glass tray after washing the porous material and before adding the porous material to the manganese salt solution.

In certain exemplary embodiments, the step of drying the porous material is performed at room temperature, 25-50° C., 50-75° C., 75-100° C., 100-125° C., 125-150° C., 150-175° C., 175-200° C. or higher than 200° C. In certain exemplary embodiments, the step of drying the porous material is performed at 25-30° C., 30-35° C., 35-40° C., 40-45° C., 45-50° C., 50-55° C., 55-60° C., 60-65° C., 65-70° C., 70-75° C., 75-80° C., 80-85° C., 85-90° C., 90-95° C., 95-100° C., 100-105° C., 105-110° C., 110-115° C., 115-120° C., 120-125° C., 125-130° C., 130-135° C., 135-140° C., 140-145° C., 145-150° C., 150-155° C., 155-160° C., 160-165° C., 165-170° C., 170-175° C., 175-180° C., 180-185° C., 185-190° C., 190-195° C. or 195-200° C. In some embodiments, the step of drying the porous material is performed at approximately 130° C. In some embodiments, the step of drying the porous material is performed for about 1.5 hours. In some embodiments, the step of drying the porous material is performed overnight.

In certain exemplary embodiments, the manganese salt is manganese (II) acetate, manganese sulfate, manganese (III) acetate, manganese (III) acetylacetonate, manganese chloride, or manganese (II) nitrate.

In certain exemplary embodiments, the process further comprises weighing the dried porous material and the manganese salt before adding the porous material. In some embodiments, the weight of the manganese salt is 0.48 times the weight of the dried porous material.

In certain exemplary embodiments, the process further comprises stirring the mixture after adding the porous material to the manganese salt solution. In some embodiments, the step of stirring the mixture is performed for less than 1 hour. In some embodiments, the step of stirring the mixture is performed for at least 1 hour. In some embodiments, the step of stirring the mixture is performed for 1-20 hours, 1-15 hours, 1-10 hours or 1-5 hours. In some embodiments, the step of stirring the mixture is performed for 5-20 hours, 5-15 hours or 5-10 hours. In some embodiments, the step of stirring the mixture is performed for 10-20 hours or 10-15 hours. In some embodiments, the step of stirring the mixture is performed for approximately 15 hours. In some embodiments, the step of stirring the mixture is performed overnight.

In certain exemplary embodiments, the step of stirring the mixture is replaced by the step of letting the mixture stand.

In certain exemplary embodiments, the process further comprises filtering the porous material after adding the porous material to the manganese salt solution. In some embodiments, the process further comprises drying the filtered porous material. In some embodiments, the step of filtering is performed using a fabric filter bag.

In certain exemplary embodiments, the step of calcinating the porous material is performed at 100-110° C., 110-120° C., 120-130° C., 130-140° C., 140-150° C., 150-160° C., 160-170° C., 170-180° C., 180-190° C., 190-200° C., 200-210° C., 210-220° C., 220-230° C., 230-240° C., 240-250° C., 250-260° C., 260-270° C., 270-280° C., 280-290° C., 290-300° C., 310-320° C., 320-330° C., 330-340° C., 340-350° C., 350-360° C., 360-370° C., 370-380° C., 380-390° C., 390-400° C., 410-420° C., 420-430° C., 430-440° C., 440-450° C., 450-460° C., 460-470° C., 470-480° C., 480-490° C. or 490-500° C. In certain exemplary embodiments, the step of calcinating the porous material is performed at 100-120° C., 120-140° C., 140-160° C., 160-180° C., 180-200° C., 200-220° C., 220-240° C., 240-260° C., 260-280° C., 280-300° C., 300-320° C., 320-340° C., 340-360° C., 360-380° C., 380-400° C., 400-420° C., 420-440° C., 440-460° C., 460-480° C. or 480-500° C. In some embodiments, the step of calcinating the porous material is performed at 100-150° C., 150-200° C., 200-250° C., 250-300° C., 300-350° C., 350-400° C., 400-450° C. or 450-500° C. In some embodiments, the step of calcinating the porous material is performed at approximately 100° C., 200° C., 300° C., 400° C. or 500° C. In some embodiments, the step of calcinating the porous material is performed at 500° C. or more. In some embodiments, the step of calcinating the porous material is performed at less than 100° C.

In certain exemplary embodiments, the process further comprises cooling down the porous materials to room temperature after calcinating the porous material.

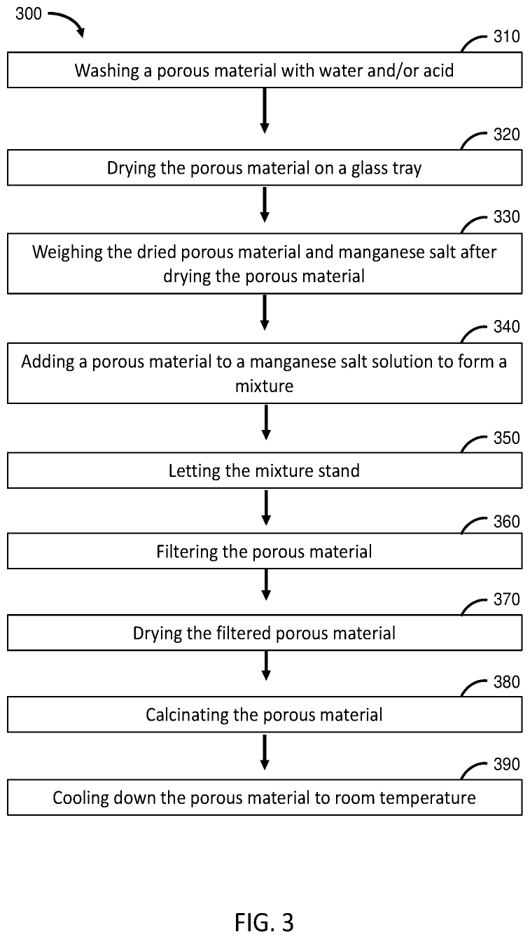

In the embodiment of FIG. 3, a method 300 for preparing a catalyst composition is illustrated. The steps are in sequential order, starting from Block 310 to 390.

The first step Block 310 states washing a porous material with water and/or acid. By way of example, the porous material is aluminum zeolite, aluminum oxide, or silica gel. In some embodiments, the porous material is approximately 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g or 200 g by weight. The acid includes but is not limited to oxalic acid, sulphuric acid, tridecyl benzene sulphonic acid and hydrochloric acid,

The next step Block 320 states drying the porous material on a glass tray. In some embodiment, the step of drying the porous material on the glass tray is performed at 130° C. for 1.5 hours.

Block 330 states weighing the dried porous material and manganese salt after drying the porous material. In some embodiments, the weight of the manganese salt is 0.48 times the weight of the dried porous material. In some embodiments, the weight of the manganese salt is 0.4-0.5 times the weight of the dried porous material. In some embodiments, the weight of the manganese salt is 0.3-0.6 times the weight of the dried porous material. In some embodiments, the weight of the manganese salt is 0.2-0.8 times the weight of the dried porous material. In some embodiments, the weight of the manganese salt is 0.1-1 times the weight of the dried porous material.

Block 340 states adding a porous material to a manganese salt solution to form a mixture. In some embodiments, the manganese salt is manganese (II) acetate.

Block 350 states letting the mixture stand. In some embodiments, the step of letting the mixture stand is performed for 1-24 hours, 1-20 hours, 2-20 hours, 3-20 hours, 4-20 hours, 5-20 hours, 6-20 hours, 7-20 hours, 8-20 hours, 9-20 hours, 10-20 hours, 11-20 hours, 12-20 hours, 13-20 hours, 14-20 hours, 15-20 hours, 1-15 hours, 2-15 hours, 3-15 hours, 4-15 hours, 5-15 hours, 6-15 hours, 7-15 hours, 8-15 hours, 9-15 hours, 10-15 hours, 11-15 hours, 12-15 hours, 13-15 hours, 14-15 hours, or approximately 15 hours.

Block 360 states filtering the porous material. In some embodiments, the step of filtering the porous material is performed using a fabric filter bag that can hold the porous material within but allow the excessive solution to flow through. Various types of fabric, mesh sizes, shapes and forms of the fabric filter bag may be used. As an example, the fabric is one or more of muslin, cheesecloth, nylon, and cotton, etc. As an example, the fabric filter bag is a soup filter bag.

Block 370 states drying the filtered porous material. In some embodiments, the step of drying the filtered porous material is skipped. In other embodiments, it is included.

Block 380 states calcinating the porous material. In some embodiments, the step of calcinating the porous material is performed at approximately 300° C. In some embodiments, the step of calcinating the porous material is performed at 100-500° C., 200-400° C., 250-350° C., 275-325° C., below 500° C., below 400° C., or below 300° C.

Block 390 states cooling down the porous material to room temperature.

One exemplary embodiment provides a method of synthesizing manganese doped granular silica mineral, comprising the steps of: (a) washing the granular silica mineral with diluted water three times followed by 2M sulfuric acid; (b) preparing a solution of manganese acetate (1-20%) mass ratio); (c) transferring the granular silica mineral to the manganese acetate solution and stirring for 5 min; (d) filtering and drying the granular silica mineral under ambient conditions; (c) calcinating the granular silica mineral at 250° C. for 2 hours; and cooling it down to room temperature to obtain the manganese doped granular silica mineral.

Another exemplary embodiment provides a method of synthesizing manganese doped granular silica mineral, comprising the steps of: (a) weighing approximately 110 g Al zeolite in a 250 ml beaker; (b) drying the Al zeolite on a glass tray in 130° C. for 1.5 hours; (c) letting the Al zeolite cool down to reasonable temperature and weighing the Al zeolite; (d) weighing (0.48*zeolite dry mass) g Mn(CH₃COO)₂·4H2O in a 1000 mL beaker; (e) measuring 480 ml distilled H₂O in a 2000 ml measuring cylinder, pouring into the 1000 ml beaker and stirring with glass rod until all Mn(CH₃COO)₂·4H2O dissolves; (f) pouring Al zeolite into the solution and letting the mixture stand for 15 hours; (g) filtering the solution through fabric filter bag; (h) pouring the filtered Al zeolite into a glass tray and roughly spreading the Al zeolite evenly on the tray; (i) calcinating the Al zeolite in a furnace at 300° C. for 2 hours; (j) letting the Al zeolite cool to room temperature, storing the zeolite in resealable plastic bags and labeling batch number, reference number(s) and conditions on the bag.

### Air Purification System

One embodiment provides an air purification system comprising: an air blower; at least one chamber having an outlet for air to flow out of the chamber; and a catalyst composition.

In certain exemplary embodiments, the air purification system has a removal efficiency for formaldehyde in the chamber of at least 95% within 20 minutes. In some embodiments, the air purification system removes formaldehyde in the chamber at a rate such that the total concentration of formaldehyde in the chamber is 1-2 ppm within 20 minutes.

In certain exemplary embodiments, the air purification system further comprises at least one VUV lamp. In some embodiments, the VUV lamp has an output of 4.7 W with a wavelength of 185 nm or 29.7 W with a wavelength of 254 nm. In some embodiments, the air purification system further comprises an ozone/ion generator. In some embodiments, the air purification system further comprises an oxidant generator wherein the oxidant generator uses high voltage difference between electrodes to generate a mixture of oxidants including one or more ions, ozone radicals or hydroxyl radicals.

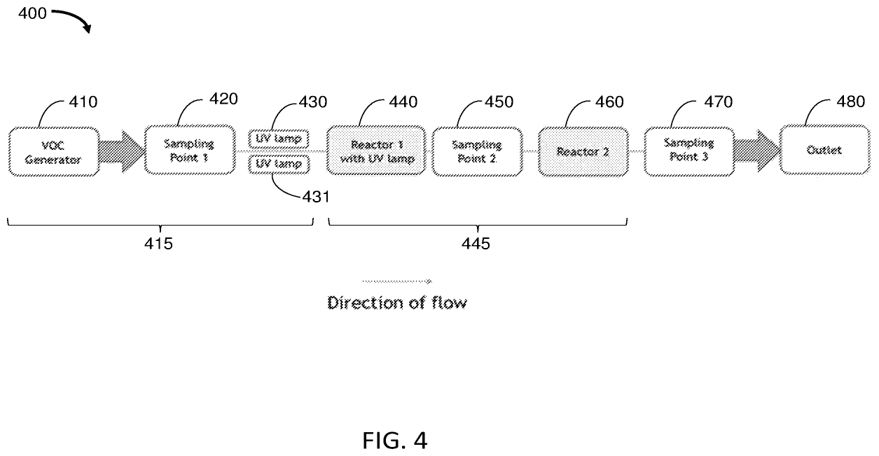

With reference FIG. 4, one example embodiment provides a volatile organic compound (VOC) removal testing unit 400 as the air purification system and its components for removing VOC. An inlet route 415 of the VOC removal testing unit 400 comprises a VOC generator 410, a sampling point 1 420 and VUV lamps 430 and 431. In some embodiments, the length of the inlet route is 4.45 m. The VOC generator 410 generates VOC. In certain embodiments of the VOC generator 410, formaldehyde with an initial concentration of 5.0 ppm is generated by bubbling a stream of compressed air into an impinger containing formalin solution with 40% formaldehyde by volume. The sampling point 1 420, a sampling point 2 450 and a sampling point 3 470 are places where air concentration of formaldehyde is measured. In some embodiments of the sampling points, the air concentration of formaldehyde is measured by using a Formaldemeter™ Htv handheld real-time meter. The VUV lamps 430 and 431 are long VUV lamps. In some embodiments, the VUV lamps 430 and 431 produce VUV which comprises about 8% 185 nm and 90% 254 nm UV. In some embodiments, the VUV produced by the VUV lamps 430 and 431 has a photon energy of 6.7 eV. In some embodiments, the VUV lamps have model no: GZW90D15Y and have a 185 nm UV output of 4.7 W and a 254 nm UV output of 29.7 W at a 1.0 m distance. In some embodiments, the length of the sampling point 1 420 and the VUV lamps 430 and 431 altogether is 1.85 m.

In some embodiments, a reactor 445 comprises a reactor 1 440 with VUV lamps, the sampling point 2 450 and a reactor 2 460. In some embodiments, the length of the reactor 445 is 1.85 m. The reactor 1 440 with VUV lamps comprises a filter filled with manganese-based catalyst composition and short VUV lamps. The short VUV lamps inside the reactor 1 440 are turned on and warmed up for 5 minutes for a VOC removal testing. In some embodiments, the length of the reactor 1 440 is 0.7 m and the length of the sampling point 2 450 is 0.45 m. The reactor 2 460 is the reactor without the VUV lamps. In some embodiments, the length of the reactor 2 460 is 0.7 m. An outlet 480 is located next to the sampling point 3 470. In some embodiments, airflow rate at the inlet route 415 and the outlet 480 of the testing unit 400 are also measured. In some embodiments, a fan speed inside the reactor 1 440 and the reactor 2 460 is set at an airflow rate of 1.5 m/s for the inlet route 415 and outlet 480 of the testing unit 400.

In certain embodiments, the air purification system further comprises an oxidant generator wherein the oxidant generator uses high voltage difference between electrodes to generate a mixture of oxidants including one or more ions, ozone radicals, and hydroxyl radicals.

In certain embodiments, the air purification system is a grossing station, a fume hood, a personal wearable air purification device, or a portable in-room system.

In certain embodiments, the grossing station is a place for specimen preparation and is used to measure, wash, dissect, and magnify the view of the specimen while dictating notes and filtering fumes in one stand-alone workstation. In some embodiments, a structure of the grossing station can be modified depending on its purpose, wherein the modified grossing station includes floor standing grossing stations and bench top grossing stations. In some embodiments, a catalyst composition described herein is placed on the grossing station such that the air circulating through the grossing station is filtered and VOCs are effectively removed. One of skill in the art would understand the details of the specific components of the grossing station. In some embodiments, the catalyst composition is placed at different locations of the grossing station according to the purpose of the grossing process.

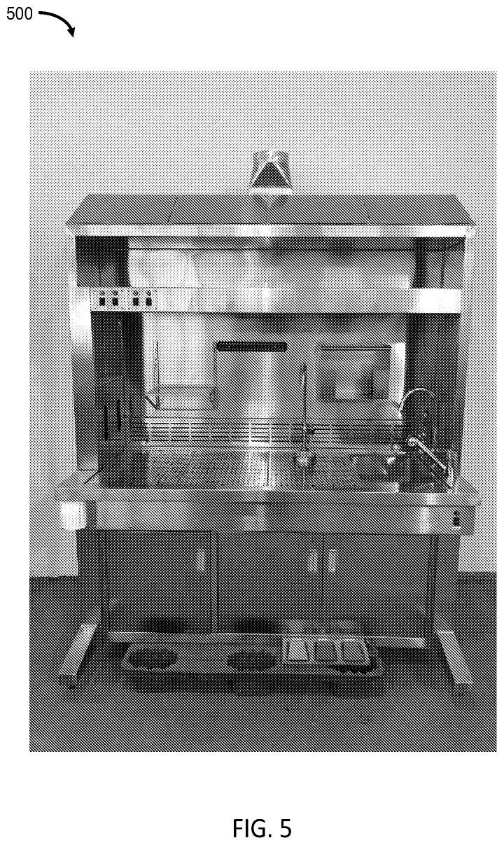

FIG. 5 is a photo showing one exemplary embodiment of the grossing station 500.

### NUMBERED EMBODIMENTS

1. A catalyst composition comprising:
a porous material having a plurality of nanopores; and
manganese oxides comprising manganese in an amount of about 0.1-50% by weight of the total catalyst composition.

2. A catalyst composition comprising:
a porous material having a plurality of nanopores and comprising one or more of silicon dioxide, aluminum oxide and zeolite; and
manganese oxides comprising manganese in an amount of about 0.1-50% by weight of the total catalyst composition,
wherein a nano sheet of the manganese oxides is formed on the porous material.

3. The catalyst composition of embodiment 1 or 2, wherein the manganese oxides form one or more clusters on the porous material.

4. The catalyst composition of any one of embodiments 1-3, wherein the manganese oxides are selected from the group consisting of MnO, MnO₂, MnO₃, Mn₃O₄ and Mn₂O₃.

5. The catalyst composition of any one of embodiments 1-4, wherein the porous material is aluminum oxide.

6. The catalyst composition of embodiment 5, wherein the aluminum oxide comprises one or more amorphous aluminum oxide, crystalline aluminum oxide, activated aluminum oxide.

7. A process for preparing the catalyst composition of any one of embodiments 1-6, comprising:
adding a porous material to a manganese salt solution to form a mixture; and
calcinating the porous material;
wherein at least one nano sheet of manganese oxides is formed on the porous material.

8. The process for preparing the catalyst composition of any one of embodiments 1-6, comprising:
washing the porous material with water and/or acid;
adding a porous material to a manganese salt solution to form a mixture; and
calcinating the porous material;
wherein at least one nano sheet of manganese oxides is formed on the porous material.

9. The process for preparing the catalyst composition of any one of embodiments 1-6, comprising:
washing the porous material with water and/or acid;
drying the porous material on a glass tray;
adding a porous material to a manganese salt solution to form a mixture; and
calcinating the porous material;
wherein at least one nano sheet of manganese oxides is formed on the porous material.

10. The process for preparing the catalyst composition of any one of embodiments 1-6, comprising:
washing the porous material with water and/or acid;
drying the porous material on a glass tray;
weighing the dried porous material and the manganese salt after drying the porous material;
adding a porous material to a manganese salt solution to form a mixture; and
calcinating the porous material;
wherein at least one nano sheet of manganese oxides is formed on the porous material.

11. The process for preparing the catalyst composition of embodiment 10, wherein the weight of the manganese salt is 0.4-0.5 times the weight of the dried porous material.

12. The process for preparing the catalyst composition of any one of embodiments 1-6, comprising:
washing the porous material with water and/or acid;
drying the porous material on a glass tray;
weighing the dried porous material and the manganese salt after drying the porous material;
adding a porous material to a manganese salt solution to form a mixture;
stirring the mixture after adding the porous material to the manganese salt solution; and
calcinating the porous material;
wherein at least one nano sheet of manganese oxides is formed on the porous material.

13. The process for preparing the catalyst composition of any one of embodiments 1-6, comprising:
washing the porous material with water and/or acid;
drying the porous material on a glass tray;
weighing the dried porous material and the manganese salt after drying the porous material;
adding a porous material to a manganese salt solution to form a mixture;
stirring the mixture after adding the porous material to the manganese salt solution;
filtering the porous material;
calcinating the porous material; and
cooling down the porous material to room temperature after calcinating the porous material
wherein at least one nano sheet of manganese oxides is formed on the porous material.

14. The process for preparing the catalyst composition of any one of the preceding embodiments, wherein the porous material is zeolite, aluminum oxide, or silica gel.

15. The process for preparing the catalyst composition of embodiment 14, wherein the zeolite is aluminum zeolite, analcime, chabazite, clinoptilolite, heulandite, phillipsite, stilbite, or natrolite.

16. The process for preparing the catalyst composition of embodiments 9-15, wherein the step of drying the porous material is performed at room temperature, 25-50° C., 50-75° C., 75-100° C., 100-125° C., 125-150° C., 150-175° C., or 175-200° C.

17. The process for preparing the catalyst composition of embodiment 16, wherein the step of drying the porous material is performed for 1.5 hours.

18. The process for preparing the catalyst composition of any one of embodiments 7-17, wherein the manganese salt is manganese (II) acetate, manganese sulfate, manganese (III) acetate, manganese (III) acetylacetonate, manganese chloride, or manganese (II) nitrate.

19. The process for preparing the catalyst composition of any one of embodiments 7-18, wherein the step of calcinating the porous material is performed at 200-220° C., 220-240° C., 240-260° C., 260-280° C., 280-300° C., 300-320° C., 320-340° C., 340-360° C., 360-380° C., or 380-400° C.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Formaldehyde Removal Test

Procedure: A grossing station, equipped with the granular silica mineral doped with manganese oxides, was placed in a 30 m³ chamber. Formaldehyde was injected into the chamber by a syringe onto a hot plate until an initial formaldehyde concentration of 1.63 ppm was achieved. The air blower inside the grossing station was turned on and the concentration of formaldehyde was recorded. The formaldehyde removal efficiency was deduced by the following:

$$Removal efficiency = \frac{{C}_{0} - {C}_{20}}{{C}_{0}} \times 100 \%$$

Result: It was found that the removal efficiency of 95% was attained after 20 minutes.

### Example 1: Formaldehyde Removal Test Supplementary Procedure

VUV is produced by VUV lamp (about 8% 185 nm and 90% 254 nm) for activation of the catalyst composition. This VUV corresponds to a photon energy of 6.7 eV and is strongly absorbed by atmospheric oxygen. The VUV dissociates water molecules to generate hydroxyl radicals:

H₂O+hv (185 nm)→·OH+·H

O₂₊·H→HO₂·

HO₂·+·H→H₂O₂

H₂O₂→hv (254 nm)→2·OH

The VUV further dissociates VOCs with low bond dissociation energy.

### Result

TABLE 1-1
Removal Efficiency without Filtering Materials

| | | | | | Removal |
|---|---|---|---|---|---|
| | Short VUV | Short VUV | Long VUV | Long VUV | Efficiency |
| | Lamp A | Lamp B | Lamp A | Lamp B | (%) |
| 1 | | | | On | 17.90 |
| 2 | On | On | | | 16.25 |
| 3 | On | On | | On | 15.25 |
| 4 | On | On | On | On | 14.43 |
| 5 | | | On | On | 14.38 |
| 6 | On | On | On | | 14.19 |
| 7 | On | | | | 12.85 |
| 8 | | On | | | 10.39 |
| 9 | | | On | | 8.18 |
| 10 | | | | | 1.02 |

TABLE 1-2
Removal Efficiency with Filtering Materials

| | | | | | Removal |
|---|---|---|---|---|---|
| | Short VUV | Short VUV | Long VUV | Long VUV | Efficiency |
| | Lamp A | Lamp B | Lamp A | Lamp B | (%) |
| 1 | On | | | | 95.21 |
| 2 | | On | | | 95.11 |
| 3 | On | On | On | On | 94.62 |
| 4 | | | On | | 93.02 |
| 5 | On | On | On | | 91.86 |
| 6 | On | On | | On | 90.47 |
| 7 | | | | | 88.54 |
| 8 | On | On | | | 86.86 |
| 9 | | | | On | 85.69 |
| 10 | | | On | On | 82.74 |

TABLE 1-3
Lifetime Test for Removal Efficiency

| | | | Removal |
|---|---|---|---|
| Date | Inlet (ppm) | Outlet (ppm) | Efficiency (%) |
| 08 Feb | 10.84 | 0.233 | 97.85 |
| 09 Feb | 10.95 | 0.239 | 97.82 |
| 10 Feb | 10.95 | 0.236 | 97.84 |
| 11 Feb | 10.92 | 0.314 | 97.12 |
| 12 Feb | 10.88 | 0.344 | 96.84 |
| 13 Feb | 10.82 | 0.141 | 98.70 |
| 14 Feb | 10.77 | 0.352 | 96.73 |

### Example 2: Enhanced NCCO Performance Test of the Manganese Doped Catalyst for HCHO Removal

### Instrumentation:

VUV lamps: Model no: GZW90D15Y-U429 at 1.0 m distance with UV output of 185 nm and 254 nm output of 4.7 W and 29.7 W, respectively. Air concentration of formaldehyde was measured by using a Formaldemeter™ Htv handheld real-time meter Formaldehyde with an initial concentration of 5.0 ppm was generated by bubbling a stream of compressed air into an impinger containing formalin solution with 40% formaldehyde by volume.

### Preparation Procedure Before the Experiment:

The 2 VUV lamps inside reactor 1 were turned on and warmed up for 5 minutes

### Airflow Rate of the Testing Unit

Airflow rate at the inlet and outlet of the system were measured

The fan speed inside the 2 reactors was set at an airflow rate of 1.5 m/s for both inlet and outlet of the system

### Background VOC Measurement

The VOC concentration at the background ambient air, inlet and outlet of the system were measured in real-time by PID VOC [RAE Systems (HONEYWELL)]

### Testing Procedure for the Formaldehyde Removal by Manganese Doped Catalyst Under VUV

A stream of compressed air was passed through the impinger for 5 min until a steady formaldehyde concentration of 10 ppm was generated. The two VUV Lamps inside the reactor 1 were turned on for 5 mins using 3 L of 13X zeolite pellets with a diameter of 4 mm. The formaldehyde concentration was measured at both sampling point 1 and sampling point 2.

The formaldehyde removal efficiency was calculated according to the following equation:

$$Removal efficency = \frac{{[ H C H O ]}_{sampling point 1} - {[ H C H O ]}_{sampling point 2}}{{[ H C H O ]}_{sampling point 1}} \times 100 \%$$

The above experimental procedures were repeated while replacing the 13X zeolite pellets by 3 L of manganese doped catalyst with a diameter of 3 mm

### Result

The formaldehyde removal efficiency using 13X zeolite (conventional NCCO): 61%. The formaldehyde removal efficiency using manganese doped catalyst (Enhanced NCCO): 96%

### Example 3: Testing Procedure for the Robustness of the Manages Doped Catalyst Under VUV

A stream of compressed air was passed through the impinger for 5 min until a steady formaldehyde concentration of 30 ppm was generated. The two VUV Lamps inside the reactor 1 were turned on for 5 mins using 3 L of manganese doped catalyst with a diameter of 3 mm. The initial concentration of formaldehyde was measured at both sampling point 1 and sampling point 2. The experiment was continued for 14 days and measurement of formaldehyde was carried out at both sampling point 1 and sampling point 2.

The formaldehyde removal efficiency was calculated according to the following equation:

$$Removal efficency = \frac{{[ H C H O ]}_{sampling point 1} - {[ H C H O ]}_{sampling point 2}}{{[ H C H O ]}_{sampling point 1}} \times 100 \%$$

### Result

The formaldehyde removal efficiency using manganese doped catalyst (Enhanced NCCO) after 14 days: 98.5%

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

The invention claimed is:

1. A catalyst composition comprising:
a porous material having a plurality of nanopores and comprising one or more of silicon dioxide, aluminum oxide and zeolite; and
manganese oxides comprising manganese in an amount of about 0.1-50% by weight of the total catalyst composition.

2. The catalyst composition of claim 1, wherein the manganese oxides form one or more clusters on the porous material.

3. The catalyst composition of claim 1, wherein the manganese has an oxidation state of +2, +3, +4, +5 or +6.

4. The catalyst composition of claim 1, wherein the manganese oxides are selected from the group consisting of MnO, MnO₂, MnO₃, Mn₃O₄ and Mn₂O₃.

5. The catalyst composition of claim 1, wherein the manganese is equal to or less than 20% by weight of the total catalyst composition.

6. The catalyst composition of claim 5, wherein the manganese is 1-20% by weight of the total catalyst composition.

7. The catalyst composition of claim 6, wherein the manganese is 4-20% by weight of the total catalyst composition.

8. The catalyst composition of claim 1, wherein the zeolite comprises one or more of aluminum oxides and silicon oxides.

9. The catalyst composition of claim 1, wherein the porous material is aluminum oxide.

10. The catalyst composition of claim 9, wherein the aluminum oxide comprises one or more of amorphous aluminum oxide, crystalline aluminum oxide, activated aluminum oxide.

11. The catalyst composition of claim 1 wherein the diameter of the nanopores is 0.6 nm-20 angstrom.

12. The catalyst composition of claim 11, wherein the diameter of the nanopores is 0.2-0.4 nm.

13. The catalyst composition of claim 1, wherein the porous material is provided in the form of a granule.

14. The catalyst composition of claim 13, wherein the diameter of each granule is 3-5 mm.

15. The catalyst composition of claim 1, wherein the manganese oxides are added to the granules via doping, ion exchange or deposition.

16. The catalyst composition of claim 1, wherein the manganese oxides have total bandgap energy of 2.0 eV to 3.75 eV.

17. A method of decomposing formaldehyde comprising the steps of:
(a) activating the catalyst composition of claim 1 with Vacuum Ultraviolet (VUV) to generate an activated catalyst composition comprising reactive oxygen species (ROS); and
(b) subjecting the activated catalyst composition comprising the ROS to formaldehyde to form carbon dioxide and water.

18. The method of claim 17, wherein the VUV is generated by a VUV lamp having an output of 4.7 W with a wavelength of 185 nm or 29.7 W with a wavelength of 254 nm.

19-54. (canceled)

55. An air purification system comprising:
an air blower;
at least one chamber having
an outlet for air to flow out of the chamber; and
a catalyst composition of claim 1.

56. The air purification system of claim 55, wherein the air purification system is a grossing station.

57. The air purification system of claim 55, wherein the air purification system has a removal efficiency for formaldehyde in the chamber of at least 95% within 20 minutes.

58. The purification system of claim 57, wherein the air purification system removes formaldehyde in the chamber at a rate such that the total concentration of formaldehyde in the chamber is 1-2 ppm within 20 minutes.

59. The air purification system of claim 55, further comprising at least one VUV lamp.

60. The air purification system of claim 59, wherein the VUV lamp has an output of 4.7 W with a wavelength of 185 nm or 29.7 W with a wavelength of 254 nm.

61. The air purification system of claim 55, further comprising an ozone/ion generator.

62. The air purification system of claim 55, further comprising an oxidant generator wherein the oxidant generator uses high voltage difference between electrodes to generate a mixture of oxidants including one or more of ions, ozone radicals, and hydroxyl radicals.
